(19) United States
(12) Patent Application Publication
(10) Pub. No.: US 2026/0157642 A1
(43) Pub. Date: Jun. 11, 2026

(54) MASSIVE BLOOD TRANSFUSION PREDICTION SYSTEM THROUGH INTRAOPERATIVE BLOOD MONITORING, LEARNING METHOD THEREOF AND MASSIVE BLOOD TRANSFUSION PREDICTION METHOD USING THEREOF

(71) Applicant: SEOUL NATIONAL UNIVERSITY HOSPITAL, Seoul (KR)

(72) Inventors: Seung Mi LEE, Seoul (KR); Hyeong Cheol LEE, Seoul (KR); Doyun KWON, Seoul (KR); Seung Bo LEE, Daegu (KR)

(21) Appl. No.: 19/417,285

(22) Filed: Dec. 11, 2025

(30) Foreign Application Priority Data
Dec. 11, 2024 (KR) 10-2024-0184102

(51) Int. Cl. A61B 5/021; A61B 5/00

(52) U.S. Cl. CPC A61B 5/02108; A61B 5/7203; A61B 5/7239; A61B 5/7267; A61B 5/7271

(57) ABSTRACT

A massive blood transfusion prediction system through intraoperative blood monitoring comprising: a photoplethysmogram (PPG) measurement sensor configured to non-invasively measure a photo-volumetric pulse wave of a patient through skin of the patient, a waveform generation unit configured to generate a photoplethysmogram (PPG) waveform representing a photoplethysmogram over time from a signal measured from the PPG measurement sensor, a feature extraction unit configured to extract one or more features from the PPG waveform and a massive blood transfusion prediction unit configured to generate a massive blood transfusion index numerically representing a necessity for massive blood transfusion through a pre-trained prediction model from the features.

### FIELD OF THE INVENTION

The present invention relates to a massive blood transfusion prediction system through intraoperative blood monitoring, a method for training thereof, and a massive blood transfusion prediction method using the system. More particularly, the present invention relates a massive blood transfusion prediction system through intraoperative blood monitoring that predicts whether massive blood transfusion is needed by noninvasively monitoring a patient's condition during surgery, a method for training thereof, and a massive blood transfusion prediction method using the system.

### BACKGROUND ART

Hemorrhage is a major cause of postoperative morbidity and mortality. When massive hemorrhage occurs in a patient during surgery, resuscitation through transfusion is required to stop the bleeding, restore intravascular fluids, restore organ perfusion, and minimize acidosis.

If massive hemorrhage occurs during surgery and the patient does not receive a prompt transfusion, serious complications may arise. To prepare blood products for transfusion during surgery in a timely manner, it is essential to predict the possibility of a massive blood transfusion (MT) in advance.

Recently, technologies have been developed for real-time prediction of intra-arterial blood pressure using intraoperative biosignals obtained from invasive monitoring of intra-arterial blood pressure. However, most surgeries are performed without equipment for invasive monitoring of intra-arterial blood pressure, and there is a problem that massive hemorrhage can occur even in cases where invasive monitoring is not performed.

### DESCRIPTION OF THE INVENTION

### Problem to be Solved

The technical problem to be solved by the present invention has been conceived from this point of view, and an object of the present invention is to provide a massive blood transfusion prediction system through intraoperative blood monitoring in a non-invasive manner.

Another object of the present invention is to provide a method for training the massive blood transfusion prediction system.

Yet another object of the present invention is to provide a massive blood transfusion prediction method using the massive blood transfusion prediction system.

### Means of Solving the Problem

According to an embodiment for achieving the above-described object of the present invention, a massive blood transfusion prediction system through intraoperative blood monitoring comprising: a photoplethysmogram (PPG) measurement sensor configured to non-invasively measure a photo-volumetric pulse wave of a patient through skin of the patient, a waveform generation unit configured to generate a photoplethysmogram (PPG) waveform representing a photoplethysmogram over time from a signal measured from the PPG measurement sensor, a feature extraction unit configured to extract one or more features from the PPG waveform and a massive blood transfusion prediction unit configured to generate a massive blood transfusion index numerically representing a necessity for massive blood transfusion through a pre-trained prediction model from the features. The massive blood transfusion refers to transfusing three or more units of red blood cells within one hour.

In an embodiment of the present invention, the system for predicting massive blood transfusion may further include a notification unit configured to notify whether the massive blood transfusion is required within 10 to 20 minutes through a value of the massive blood transfusion index.

In an embodiment of the present invention, the feature extracted by the feature extraction unit is a cycle duration (Tc) of the PPG waveform.

In an embodiment of the present invention, the cycle duration is calculated as a time interval between start and end points in a graph for an area under the PPG waveform.

In an embodiment of the present invention, the waveform generation unit further generates a velocity of photoplethysmogram (VPG) waveform, which is a first derivative of the PPG waveform. The feature extraction unit further extracts one or more features from the VPG waveform. The massive blood transfusion prediction unit uses two or more features among the features extracted from the PPG waveform and the VPG waveform as an input for the prediction model.

In an embodiment of the present invention, the waveform generation unit further generates an acceleration of photoplethysmogram (APG) waveform, which is a second derivative of the PPG waveform. The feature extraction unit further extracts one or more features from the APG waveform. The massive blood transfusion prediction unit uses two or more features among the features extracted from the PPG waveform and the APG waveform as an input for the prediction model.

According to an embodiment for achieving the above-described object of the present invention, a method for training a massive blood transfusion prediction system that predicts in advance a necessity for massive blood transfusion during surgery using a prediction model, the method comprising: a data collection step of collecting training data including whether and when a massive blood transfusion was performed on a patient during surgery and a PPG waveform, a data processing step of preprocessing the PPG waveform of the data and extracting features, a model training step of training the prediction model using the extracted features and a validation step of validating performance of the prediction model by calculating an area under the receiver operating characteristic curve (AUROC).

In an embodiment of the present invention, the features include at least one of: i) a systolic peak(S), ii) a start of a systolic wave (O), iii) (⅓)(S+(2*O))*(M), iv) a cycle duration (Tc), and v) an area under the waveform (au_W) of the preprocessed PPG waveform.

In an embodiment of the present invention, in the data processing step, a velocity of photoplethysmogram (VPG) waveform, which is a first derivative of the preprocessed PPG waveform, is further generated, and one or more features are further extracted from the VPG waveform. In the model training step, training is performed by using two or more features among the features extracted from the PPG waveform and the VPG waveform as an input for the prediction model.

In an embodiment of the present invention, in the data processing step, an acceleration of photoplethysmogram (APG) waveform, which is a second derivative of the preprocessed PPG waveform, is further generated, and one or more features are further extracted from the APG waveform. In the model training step, training is performed by using two or more features among the features extracted from the PPG waveform and the APG waveform may be used as an input for the prediction model.

According to an embodiment for achieving the above-described object of the present invention, a massive blood transfusion prediction method using a massive blood transfusion prediction system through intraoperative blood monitoring, comprising: a bio-signal measurement step of non-invasively measuring a photo-volumetric pulse wave of a patient through skin of the patient using a photoplethysmogram (PPG) measurement sensor, a preprocessing step of generating a photoplethysmogram (PPG) waveform representing a photoplethysmogram over time from a signal measured through the PPG measurement sensor and performing noise removal or smoothing, a feature extraction step of extracting one or more features from the preprocessed PPG waveform, and a massive blood transfusion prediction step of generating a massive blood transfusion index numerically representing a necessity for massive blood transfusion through a pre-trained prediction model from the features. The massive blood transfusion is defined as transfusing three or more units of red blood cells within one hour.

### Effects of the Invention

According to embodiments of the present invention, a prediction model through deep learning of the massive blood transfusion prediction system can help improve patient prognosis by rapidly assessing the degree of hemorrhage before a surgeon or an anesthesiologist recognizes the amount of blood loss, thereby predicting the timing when a patient requires massive blood transfusion.

In particular, even in a surgical procedure where intra-arterial blood pressure is not invasively monitored, the necessity of massive blood transfusion can be predicted in advance using only a non-invasive method, thereby significantly reducing surgical risks.

However, the effects of the present invention are not limited to the aforementioned effects, and may be variously expanded within a range that does not depart from the spirit and scope of the present invention.

### BRIEF DESCRIPTION OF THE DRAWINGS

FIG. 1 is a plan view of a massive blood transfusion prediction system through intraoperative blood monitoring according to an embodiment of the present invention.

FIG. 2 is a flowchart for explaining a method for training a massive blood transfusion prediction system through intraoperative blood monitoring according to an embodiment of the present invention.

FIG. 3 is a flowchart for explaining a massive blood transfusion prediction method using a massive blood transfusion prediction system through intraoperative blood monitoring according to an embodiment of the present invention.

FIG. 4 is a diagram for explaining data selection (a), preprocessing, feature extraction (c), input source selection (d), modeling (e), and a prediction result (f) of the massive blood transfusion prediction system according to an embodiment of the present invention.

FIG. 5 is a diagram for explaining data and performance used for training the massive blood transfusion prediction system according to an embodiment of the present invention.

FIG. 6 is a diagram for explaining performance of a prediction model according to features and combinations thereof that can be used in a massive blood transfusion prediction unit of the system for predicting massive blood transfusion according to an embodiment of the present invention.

FIG. 7 is a diagram showing an example of a real-time massive blood transfusion index (MTi) of a patient who received a massive blood transfusion during surgery.

FIG. 8 is a diagram showing a configuration of actual patient data used in an experimental example of the present invention.

FIG. 9 is a diagram showing predictive AUROC at 10 minutes, 15 minutes, and 20 minutes of a prediction model used in an experimental example of the present invention.

### DETAILED DESCRIPTION OF THE INVENTION

Hereinafter, preferred embodiments of the present invention will be described in more detail with reference to the accompanying drawings.

As the present invention may be variously modified and have various forms, specific embodiments will be illustrated in the drawings and described in detail in the text. However, this is not intended to limit the present invention to specific disclosure forms, and it should be understood to include all modifications, equivalents, or substitutes included in the spirit and technical scope of the present invention.

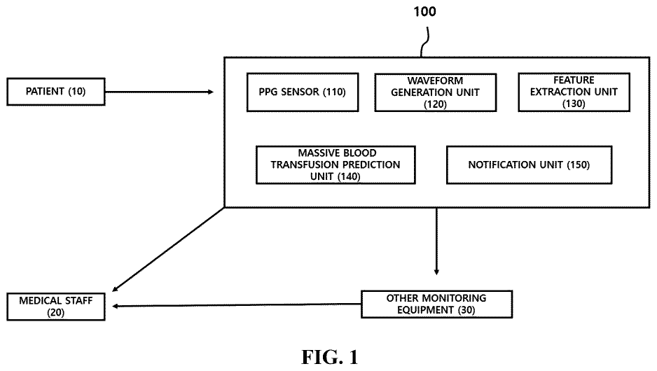

FIG. 1 is a plan view of a massive blood transfusion prediction system through intraoperative blood monitoring according to an embodiment of the present invention.

Referring to FIG. 1, the system massive blood transfusion prediction system (100) may include a PPG sensor (110), a waveform generation unit (120), a feature extraction unit (130), a massive blood transfusion prediction unit (140), and a notification unit (150).

The PPG sensor (110) is a sensor that measures changes in blood flow using light, and consists of an LED light source and a photodetector. The PPG sensor (110) may illuminate skin of a patient (10) with light, detect changes in blood flow by measuring an amount of reflected light, and generate a photoplethysmogram (PPG) measurement signal that changes over time.

For example, the PPG sensor (110) may be attached to a finger of the patient (10) to contact an inner surface of the finger, measure changes in a photo-volumetric pulse wave of the patient (10), and generate a photoplethysmogram (PPG) signal.

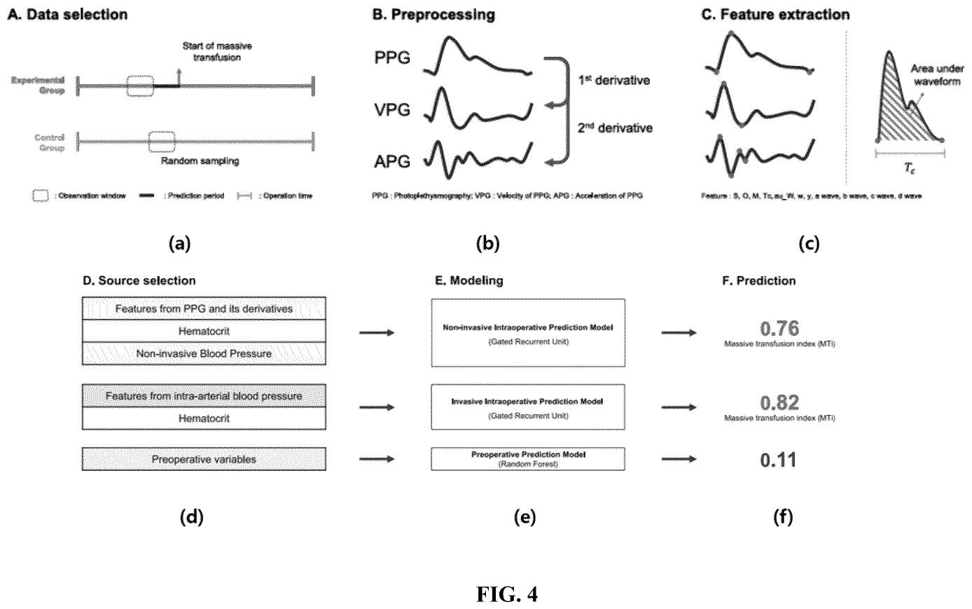

The waveform generation unit (120) receives the PPG signal from the PPG sensor (110), and after preprocessing the PPG signal, may generate waveforms required for predicting massive blood transfusion. Specifically, the waveform generation unit (120) may generate a PPG waveform representing the measured photoplethysmogram, a velocity of photoplethysmogram (VPG) waveform which is a first derivative of the PPG waveform, an acceleration of photoplethysmogram (APG) waveform which is a second derivative of the PPG waveform, and the like (refer to (b) and (c) of FIG. 4).

A Savitzky-Golay filter may be used for the preprocessing. The Savitzky-Golay filter is a digital filter used for data smoothing and is used to increase precision of data without distorting a trend of a signal of the data. The Savitzky-Golay filter may serve to reduce noise while maintaining a width and trend of the signal.

The feature extraction unit (130) may extract features from the PPG waveform, the VPG waveform, or the APG waveform from the waveform generation unit (120). By extracting features from the waveforms, high-dimensional characteristics of data can be appropriately considered and computational efficiency can be increased.

The features may be: i) a systolic peak(S), ii) a start of a systolic wave (O), iii) (⅓)(S+(2O))*(M), iv) a cycle duration (Tc), and v) an area under the waveform (au_W), which are extracted from the PPG waveform.

In the VPG waveform, i) a maximum slope during contraction (w) and ii) a minimum slope during contraction (y) may be extracted as the features.

Since all major points of the VPG waveform reflect vascular conditions, features for i) an a-wave (a), 2) a b-wave (b), 3) a c-wave (c), and 4) a d-wave (d) may be extracted from the VPG waveform. The a-wave represents an initial increase in blood flow during a systolic phase of the heart and occurs when the heart pumps blood into the aorta. The b-wave represents a section where the blood flow decreases after reaching a peak and is caused by elastic recoil of the aorta. The c-wave represents a section where the blood flow increases again, which is caused by resistance of peripheral vessels. The d-wave represents a section where the blood flow decreases again, which occurs during a diastolic phase of the heart. The b-wave (b) represents a section where blood flow decreases after reaching a peak and is caused by elastic recoil of the aorta. Said c-wave (c) represents a section where blood flow increases again, which is caused by resistance of peripheral vessels. Said d-wave (d) represents a section where blood flow decreases again, which occurs during a diastolic phase of the heart.

Meanwhile, intra-arterial blood pressure of the patient (10) may be directly measured and used to improve prediction quality of the massive blood transfusion prediction unit (140) or utilized as data for training a prediction model. When the intra-arterial blood pressure is directly measured, i) an area under arterial waveform (AUAW) for each heartbeat, ii) mean arterial pressure (MBP), iii) systolic blood pressure (SBP), iv) diastolic blood pressure (DBP), and v) heart rate (HR) may be extracted therefrom. In addition, hematocrit is measured according to a determination by an anesthesiologist, and non-invasive blood pressure (NIBP) may be measured and utilized as a feature or for comparison.

The massive blood transfusion prediction unit (140) may predict a timing when the massive blood transfusion is necessary for the patient (10) by using various features extracted from the feature extraction unit (130). Here, massive blood transfusion (MT) is defined as transfusing three or more units of red blood cells within one hour. A volume of one unit of the red blood cells may be 200 to 240 ml, and upon transfusing one unit of the red blood cells, a hemoglobin level of the patient (10) may increase by approximately 1 g/dl.

The massive blood transfusion prediction unit (140) defines and predicts the necessity for the massive blood transfusion as a variable MT, and may include a deep learning-based prediction model that predicts intraoperative MT within 10 minutes.

The prediction model was implemented by the following method.

Patients targeted for obtaining training data consisted of 18,135 patients who underwent surgery at Seoul National University Hospital (SNUH) and 621 patients who underwent surgery at Boramae Medical Center (BMC) for external validation.

Massive blood transfusion (MT) was defined as administering three or more units of red blood cells within one hour, and cases where information on the timing of transfusion was inaccurate or the amount of transfusion did not meet the definition of massive blood transfusion (MT) were excluded.

In selection and preprocessing of data, preoperative variables such as demographic information of patients, complications, and preoperative test results from electronic health records were collected to develop a preoperative prediction model, and several intraoperative parameters were collected as follows to develop an intraoperative prediction model.

1) features of PPG and derivative variables (VPG, APG), 2) features of intra-arterial blood pressure, 3) NIBP (non-invasively measured blood pressure), and 4) intraoperative hematocrit levels obtained during surgery.

Since the features have been described in detail in the description of the feature extraction unit (130), redundant descriptions will be omitted.

An observation window and a prediction period for said features are set. Intraoperative features extracted from a single 10-minute observation window were used as an input for the prediction model. The observation window was set differently depending on patients who received massive blood transfusion (MT) (experimental group) and patients who did not (control group) (refer to (c) of FIG. 4).

In the experimental group, the intraoperative features were extracted 20 to 10 minutes before the start of massive blood transfusion (MT). Through this method, an important indicator for predicting the necessity for massive blood transfusion (MT) can be captured. In the control group, where massive blood transfusion did not occur during surgery, a randomly selected period throughout the entire surgical duration was extracted as the observation window. This approach is intended to provide a consistent and unbiased sampling method across all non-critical periods. Additionally, to compare optimal prediction times, prediction periods were set differently to 10 minutes, 15 minutes, and 20 minutes before the massive blood transfusion for comparison.

The prediction model is an artificial intelligence prediction model, which calculates the necessity (risk) for massive blood transfusion 10 minutes later during surgery to derive a massive blood transfusion variable, i.e., an MT variable. The prediction model may receive the following data as an input (refer to (d), (e), and (f) of FIG. 4)

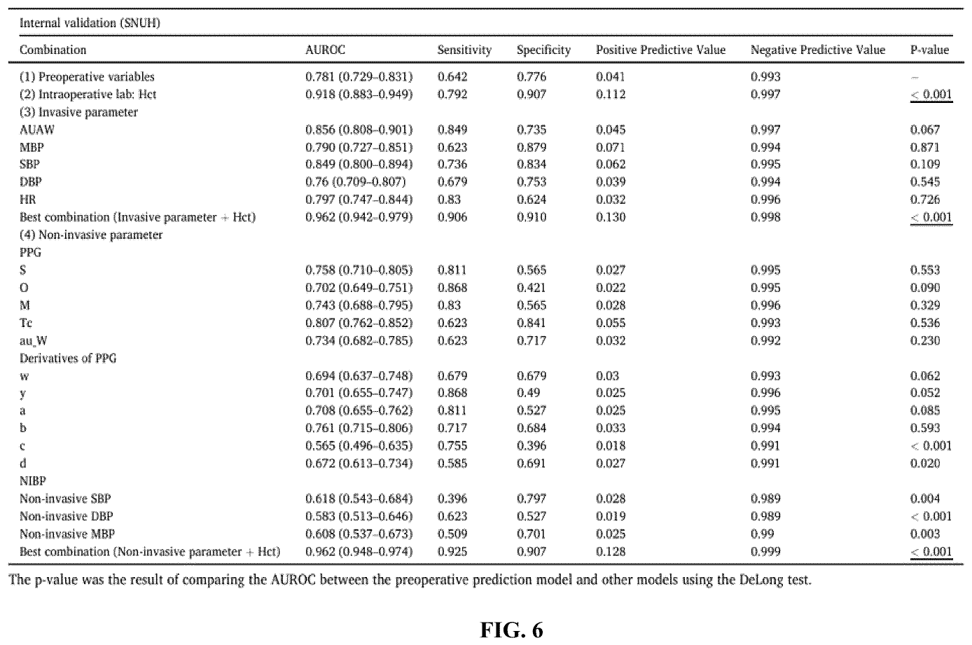

Even when only non-invasive monitoring information using 1) features of PPG and 2) intraoperative hematocrit levels obtained during surgery was used as the input, it was possible to predict massive blood transfusion with sufficient accuracy (AUROC=0.96). It was confirmed that sufficiently accurate prediction could be performed compared to a prediction model (AUROC=0.96) that additionally receives features of intra-arterial blood pressure measured through an invasive method (refer to FIG. 6).

The prediction model can calculate the MT risk after 10 minutes. Since such algorithms can learn complex hierarchical and time-series representations, deep learning-based algorithms such as a Recurrent Neural Network (RNN), a Long Short-Term Memory (LSTM), and a Gated Recurrent Unit (GRU) may be used to learn these features.

Meanwhile, as a comparative example, it can be seen that satisfactory results were not obtained (AUROC=0.78) when preoperative variables, such as demographic information, complications, and preoperative laboratory test results, were used as input data. Here, algorithms such as Logistic Regression (LR), Random Forest (RF), XGBoost (XGB), and Light Gradient Boosting Machine (LGBM) were used for the preoperative prediction model (refer to FIG. 6).

The notification unit (150) may notify the medical staff (20) of the necessity for massive blood transfusion directly or through other monitoring equipment (30) when the necessity for massive blood transfusion within a specific time is predicted by the massive blood transfusion prediction unit (140). For example, a warning display regarding the necessity for massive blood transfusion may be displayed on a display device, or a warning sound or an announcement message regarding the necessity for massive blood transfusion may be output through a speaker. Accordingly, it is possible to make the medical staff focusing on surgery immediately aware of the necessity for massive blood transfusion and to allow for appropriate preparation, thereby improving surgical safety.

The other monitoring equipment (30) is a device for monitoring various conditions of the patient, and can detect the necessity for massive blood transfusion in real time by using information received through the notification unit (150) along with various vital signs of the patient.

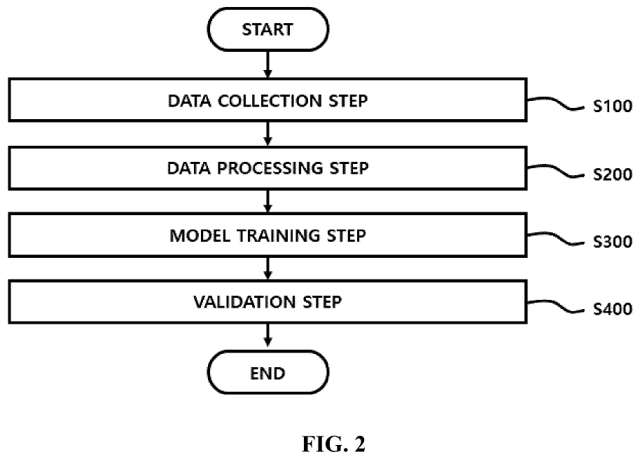

FIG. 2 is a flowchart for explaining a method for training a massive blood transfusion prediction system through intraoperative blood monitoring according to an embodiment of the present invention.

Referring to FIG. 2, the method for training the massive blood transfusion prediction system includes a data collection step (S100), a data processing step (S200), a model training step (S300), and a validation step (S400).

In the data collection step (S100), data of patients during surgery are collected to obtain training data for the system for predicting massive blood transfusion.

In selecting the patient data, preoperative variables such as demographic information, complications, and preoperative test results from electronic health records are collected to develop a preoperative prediction model, and various intraoperative parameters are collected as follows to develop an intraoperative prediction model.

Meanwhile, for model training, features extracted from plethysmography collected at 500 Hz and intraoperative hematocrit were additionally used, and i) an area under arterial waveform (AUAW) for each heartbeat, ii) mean arterial pressure (MBP), iii) systolic blood pressure (SBP), iv) diastolic blood pressure (DBP), and v) heart rate (HR) may be extracted from the measured intra-arterial blood pressure. In addition, hematocrit is measured according to a determination by an anesthesiologist, and non-invasive blood pressure (NIBP) was measured and utilized as a feature or for comparison.

1) Features of PPG and derivative variables (VPG, APG), 2) features of intra-arterial blood pressure, 3) NIBP (non-invasively measured blood pressure), and 4) intraoperative hematocrit levels (hct) obtained during surgery.

Since the features have been described in detail in the description of the feature extraction unit of FIG. 1, redundant descriptions will be omitted.

In the data processing step (S200), the PPG waveform and derivative variables thereof may be pre-processed using a Savitzky-Golay filter before feature extraction. Features are extracted from the pre-processed data, and specifically, the following features may be extracted from each data.

Features extracted from the PPG waveform include i) a systolic peak(S), ii) a start of a systolic wave (O), iii) (⅓)(S+(2*O))*(M), iv) a cycle duration (Tc), and v) an area under the waveform (au_W).

In the VPG waveform, i) a maximum slope during contraction (w) and ii) a minimum slope during contraction (y) may be extracted as the features.

Since all major points of the VPG waveform reflect vascular conditions, features for i) an a-wave (a), 2) a b-wave (b), 3) a c-wave (c), and 4) a d-wave (d) may be extracted from the VPG waveform.

In the model training step (S300), the features extracted in the data processing step (S200) may be used to train the prediction model of the system for predicting massive blood transfusion.

By setting a single 10-minute observation window for the features, the intraoperative features extracted from the single 10-minute interval observation window may be used as an input for the prediction model. The observation window may be set differently depending on whether the patient received massive blood transfusion (MT) (experimental group) or did not receive massive blood transfusion (MT) (control group).

In the validation step (S400), prediction performance of the prediction model trained through various methods may be measured and validated for each feature combination by calculating an area under the receiver operating characteristic curve (AUROC).

To design an optimal prediction model, the AUROCs of the preoperative prediction model and the intraoperative prediction model using various features and input variables were compared using the DeLong test. Sensitivity, specificity, positive predictive value (PPV), negative predictive value (NPV), and the F1 score at an optimal threshold that maximizes the sum of sensitivity and specificity (i.e., Youden's J statistic) were used to predict the performance of the proposed method. The optimal threshold for classification was determined using Youden's J statistic within an internal validation set. Subsequently, the performance of the model was evaluated in an external validation set using the defined threshold.

A 95% confidence interval (CI) of the AUROC was calculated using 2,000 bootstrapping resamplings, and such a risk score was defined as a massive blood transfusion index (MTi). All analyses were performed using R (version 4.3.1) and Python (version 3.8.10).

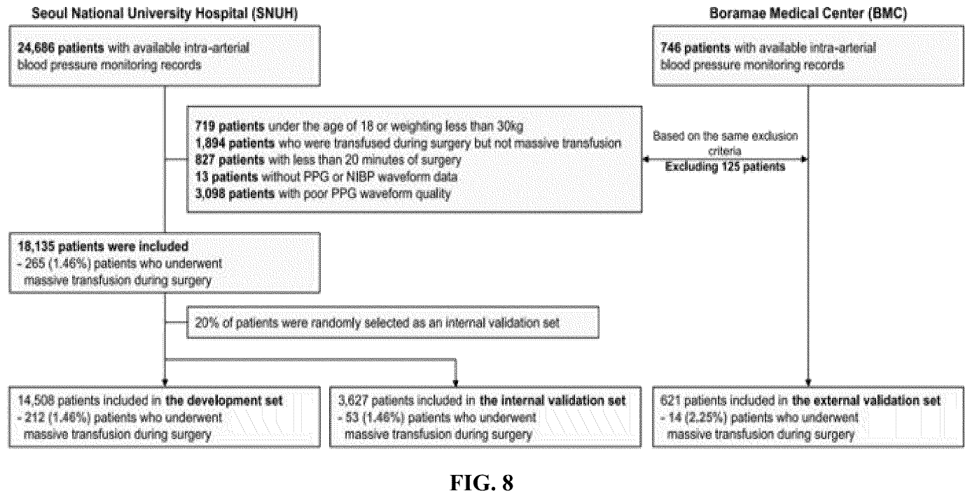

Specifically, the patient data used for the experiment include patients who underwent surgery at Seoul National University Hospital (SNUH) between August 2016 and December 2019. Among them, patients with intra-arterial blood pressure monitoring records (n=24,686) were included (refer to FIG. 8). The following surgical cases were excluded from the analysis: 1) patients under 18 years of age, 2) patients with low weight (less than 30 kg), 3) patients who received intraoperative transfusion but not MT, 4) patients with short surgery time (less than 20 minutes), and 5) patients without PPG or non-invasive blood pressure (NIBP) monitoring information or with poor signal quality. Patients who underwent surgery at SNUH were randomly divided into 80% for model development and 20% for model validation. For external validation, patients with intra-arterial blood pressure monitoring records (n=746) at Boramae Medical Center (BMC) between 2020 and 2021 were identified. The same exclusion criteria as SNUH were applied to BMC.

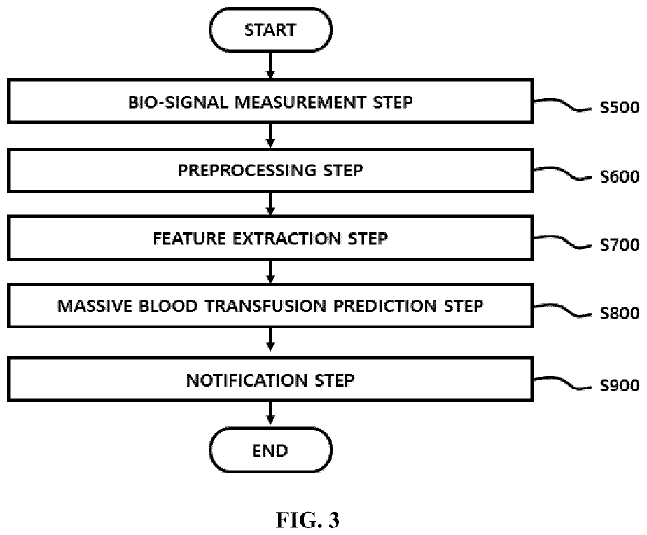

FIG. 3 is a flowchart for explaining a massive blood transfusion prediction method using a massive blood transfusion prediction system through intraoperative blood monitoring according to an embodiment of the present invention.

Referring to FIG. 3, the massive blood transfusion prediction method using a massive blood transfusion prediction system through intraoperative blood monitoring can predict the necessity for massive blood transfusion within a predetermined time based on real-time intraoperative monitoring data using a pre-trained prediction model, and notify the medical staff.

The method for predicting massive blood transfusion through intraoperative blood monitoring may include a bio-signal measurement step (S500), a preprocessing step (S600), a feature extraction step (S700), a massive blood transfusion prediction step (S800), and a notification step (S900).

In the bio-signal measurement step (S500), a photo-volumetric pulse wave of a patient may be non-invasively measured through skin of the patient using a photoplethysmogram (PPG) measurement sensor.

In the preprocessing step (S600), a photoplethysmogram (PPG) waveform representing a photoplethysmogram over time may be generated from a signal measured through the PPG measurement sensor, and noise removal or smoothing may be performed.

In the feature extraction step (S700), one or more features may be extracted from the preprocessed PPG waveform.

In the massive blood transfusion prediction step (S800), the necessity for transfusion is predicted by generating a massive blood transfusion index that numerically represents the necessity for massive blood transfusion through the pre-trained prediction model from the features.

In the notification step (S900), the necessity for the massive blood transfusion within 10 to 20 minutes is notified through a change in the massive blood transfusion index generated in the massive blood transfusion prediction step.

Since specific data processing in each of the steps is substantially the same as the description for FIG. 1 above, redundant descriptions will be omitted.

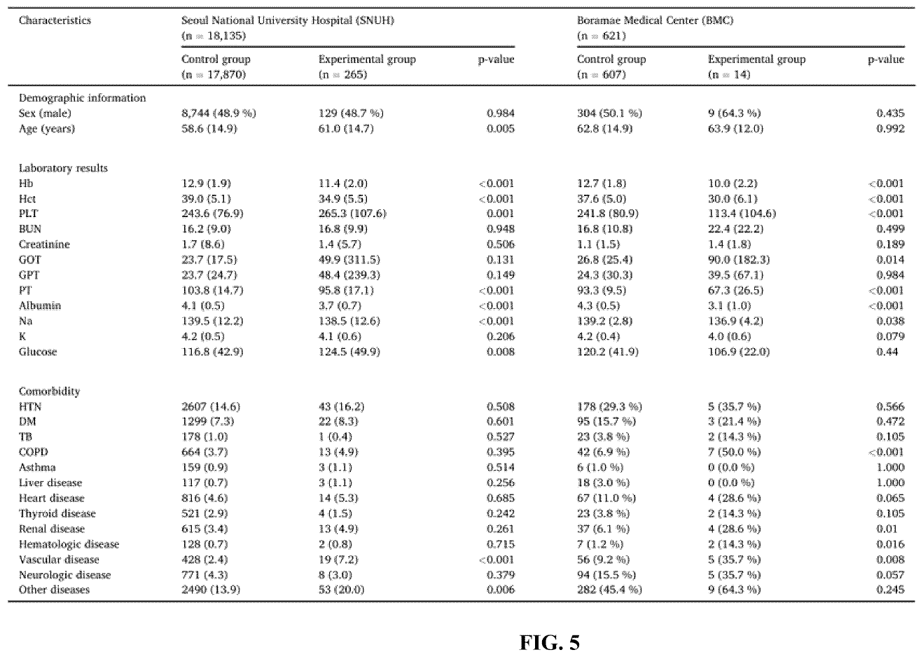

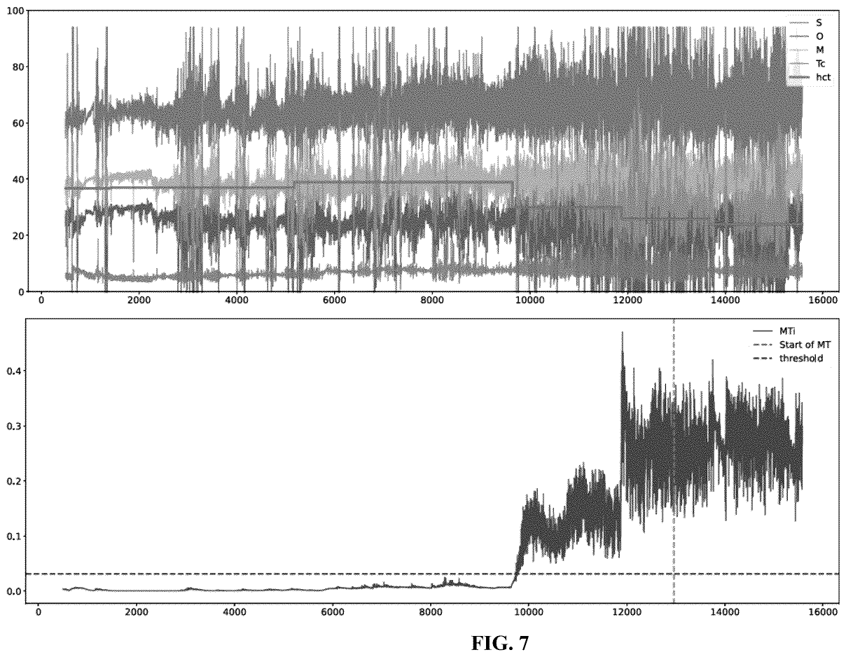

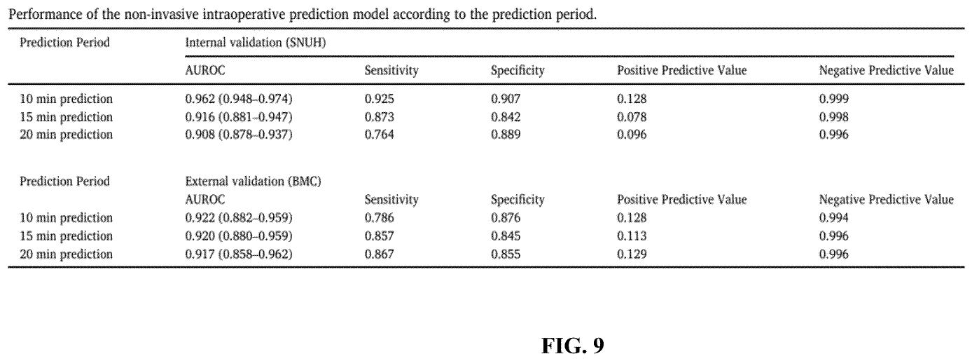

FIG. 4 is a diagram for explaining data selection (a), preprocessing, feature extraction (c), input source selection (d), modeling (e), and a prediction result (f) of the massive blood transfusion prediction system according to an embodiment of the present invention. FIG. 5 is a diagram for explaining data and performance used for training the massive blood transfusion prediction system according to an embodiment of the present invention. FIG. 6 is a diagram for explaining performance of a prediction model according to features and combinations thereof that can be used in a massive blood transfusion prediction unit of the system for predicting massive blood transfusion according to an embodiment of the present invention. FIG. 7 is a diagram showing an example of a real-time massive blood transfusion index (MTi) of a patient who received a massive blood transfusion during surgery. FIG. 8 is a diagram showing a configuration of actual patient data used in an experimental example of the present invention. FIG. 9 is a diagram showing predictive AUROC at 10 minutes, 15 minutes, and 20 minutes of a prediction model used in an experimental example of the present invention.

Referring to FIGS. 4 to 9, performances of three models (a preoperative prediction model, an invasive intraoperative prediction model, and a non-invasive intraoperative prediction model) were compared. First, the best-performing preoperative prediction model was Random Forest (RF) having an AUROC (area under the receiver operating characteristic curve) of 0.781 (95% CI, 0.729-0.831). Then, performances of the non-invasive intraoperative prediction model and the invasive intraoperative prediction model were evaluated using deep learning-based algorithms. Among various deep learning-based algorithms, Gated Recurrent Unit (GRU)—which showed an AUROC of 0.787 (95% CI, 0.736-0.837) for PPG features, 0.731 (95% CI, 0.671-0.786) for VPG features, 0.721 (95% CI, 0.663-0.767) for APG features, and 0.870 (95% CI, 0.829-0.907) for intra-arterial blood pressure features—was the best algorithm for all intraoperative feature combinations. In internal validation, hct (hematocrit) showed the best performance as a single variable with an AUROC of 0.918 (95% CI, 0.883-0.949). In the invasive intraoperative prediction model, the best combination consisted of AUAW and hct, and the AUROC was 0.962 (95% CI, 0.942-0.979). In the non-invasive intraoperative prediction model, the best combination consisted of Tc and hct, and the AUROC was 0.962 (95% CI, 0.948-0.974). The performance of external validation was maintained with an AUROC of 0.922 (95% CI, 0.882-0.959). The AUROCs of the preoperative and non-invasive intraoperative prediction models were significantly different (p<0.001). Furthermore, the non-invasive prediction model was not statistically different from the invasive prediction models of previous studies. In addition, whether the best prediction model can predict MT risk not only 10 minutes before but also 15 or 20 minutes before was investigated, and the performance of the non-invasive intraoperative prediction model according to the prediction period was similar in internal and external validation (10 minutes before vs. 15 or 20 minutes before) (refer to FIG. 9). The developed prediction model not only shows robust performance in predicting MT necessity but also can be effectively extended to broader transfusion requirements.

Meanwhile, since the prediction model shows sufficient predictability even with only the cycle duration (Tc) extracted from the PPG waveform, it may be implemented by simply using only Tc as a feature, which is an input value of the prediction model. Here, the cycle duration (Tc) is a time interval between start and end points of an area under waveform (au_W), and can be calculated using a known calculation method for calculating an area under a waveform from a waveform.

In the internal validation set, there was a 17.6-fold difference in the average MTi value between patients who received intraoperative MT and those who did not. Additionally, the difference between the two groups was 8.27 times greater in the external validation dataset. In most of the control group (patients who did not receive intraoperative MT), MTi was less than 0.01, but in most of the experimental group (patients who received intraoperative MT), MTi was >0.01. Referring to FIG. 7 together, FIG. 7 shows an example of MTi of a patient who received MT at 216 minutes after the start of surgery. The MTi increased above the MT threshold from 162 minutes after surgery.

According to embodiments of the present invention, a deep learning-based algorithm (DLA) that predicts intraoperative MT within 10 minutes using intraoperative bio-signal waveforms non-invasively using PPG can be implemented.

In various prediction models according to embodiments of the present invention, the best-performing DLA includes Tc and hct. PPG is a non-invasive optical technology commonly used to measure changes in blood volume in peripheral tissues. By analyzing PPG signals, important changes such as heart rate (HR) and blood pressure (BP) variations can be predicted. HR can be accurately estimated by analyzing PPG waveform characteristics including amplitude, pulse width, and shape. By applying machine learning algorithms to PPG data, a model capable of predicting HR changes in real time can be trained.

Similarly, PPG signals can provide insights into BP fluctuations by examining pulse transit time (PTT) derived from PPG and other factors such as arterial stiffness. PTT is the time required for an arterial pulse wave to propagate between two arterial sites and is inversely proportional to BP. Through continuous monitoring of PPG signals and the application of advanced data analysis techniques such as signal processing and deep learning, PPG-based prediction models can provide valuable information on critical changes such as the necessity for massive blood transfusion. These predictions can help in early detection of abnormalities and provide feedback for health monitoring and management.

In particular, according to embodiments of the present invention, computational efficiency can be increased and a response time of the model can be shortened by using relatively small data through a non-invasive method.

Although the present invention has been described with reference to the above embodiments, it will be understood by those skilled in the art that various modifications and changes can be made to the present invention without departing from the spirit and scope of the present invention as set forth in the following claims.

Hereinafter, preferred embodiments of the present invention will be described in more detail with reference to the accompanying drawings.

As the present invention may be variously modified and have various forms, specific embodiments will be illustrated in the drawings and described in detail in the text. However, this is not intended to limit the present invention to specific disclosure forms, and it should be understood to include all modifications, equivalents, or substitutes included in the spirit and technical scope of the present invention.

FIG. 1 is a plan view of a massive blood transfusion prediction system through intraoperative blood monitoring according to an embodiment of the present invention.

Referring to FIG. 1, the system massive blood transfusion prediction system (100) may include a PPG sensor (110), a waveform generation unit (120), a feature extraction unit (130), a massive blood transfusion prediction unit (140), and a notification unit (150).

The PPG sensor (110) is a sensor that measures changes in blood flow using light, and consists of an LED light source and a photodetector. The PPG sensor (110) may illuminate skin of a patient (10) with light, detect changes in blood flow by measuring an amount of reflected light, and generate a photoplethysmogram (PPG) measurement signal that changes over time.

For example, the PPG sensor (110) may be attached to a finger of the patient (10) to contact an inner surface of the finger, measure changes in a photo-volumetric pulse wave of the patient (10), and generate a photoplethysmogram (PPG) signal.

The waveform generation unit (120) receives the PPG signal from the PPG sensor (110), and after preprocessing the PPG signal, may generate waveforms required for predicting massive blood transfusion. Specifically, the waveform generation unit (120) may generate a PPG waveform representing the measured photoplethysmogram, a velocity of photoplethysmogram (VPG) waveform which is a first derivative of the PPG waveform, an acceleration of photoplethysmogram (APG) waveform which is a second derivative of the PPG waveform, and the like (refer to (b) and (c) of FIG. 4).

A Savitzky-Golay filter may be used for the preprocessing. The Savitzky-Golay filter is a digital filter used for data smoothing and is used to increase precision of data without distorting a trend of a signal of the data. The Savitzky-Golay filter may serve to reduce noise while maintaining a width and trend of the signal.

The feature extraction unit (130) may extract features from the PPG waveform, the VPG waveform, or the APG waveform from the waveform generation unit (120). By extracting features from the waveforms, high-dimensional characteristics of data can be appropriately considered and computational efficiency can be increased.

The features may be: i) a systolic peak(S), ii) a start of a systolic wave (O), iii) (⅓)(S+(2O))*(M), iv) a cycle duration (Tc), and v) an area under the waveform (au_W), which are extracted from the PPG waveform.

In the VPG waveform, i) a maximum slope during contraction (w) and ii) a minimum slope during contraction (y) may be extracted as the features.

Since all major points of the VPG waveform reflect vascular conditions, features for i) an a-wave (a), 2) a b-wave (b), 3) a c-wave (c), and 4) a d-wave (d) may be extracted from the VPG waveform. The a-wave represents an initial increase in blood flow during a systolic phase of the heart and occurs when the heart pumps blood into the aorta. The b-wave represents a section where the blood flow decreases after reaching a peak and is caused by elastic recoil of the aorta. The c-wave represents a section where the blood flow increases again, which is caused by resistance of peripheral vessels. The d-wave represents a section where the blood flow decreases again, which occurs during a diastolic phase of the heart. The b-wave (b) represents a section where blood flow decreases after reaching a peak and is caused by elastic recoil of the aorta. Said c-wave (c) represents a section where blood flow increases again, which is caused by resistance of peripheral vessels. Said d-wave (d) represents a section where blood flow decreases again, which occurs during a diastolic phase of the heart.

Meanwhile, intra-arterial blood pressure of the patient (10) may be directly measured and used to improve prediction quality of the massive blood transfusion prediction unit (140) or utilized as data for training a prediction model. When the intra-arterial blood pressure is directly measured, i) an area under arterial waveform (AUAW) for each heartbeat, ii) mean arterial pressure (MBP), iii) systolic blood pressure (SBP), iv) diastolic blood pressure (DBP), and v) heart rate (HR) may be extracted therefrom. In addition, hematocrit is measured according to a determination by an anesthesiologist, and non-invasive blood pressure (NIBP) may be measured and utilized as a feature or for comparison.

The massive blood transfusion prediction unit (140) may predict a timing when the massive blood transfusion is necessary for the patient (10) by using various features extracted from the feature extraction unit (130). Here, massive blood transfusion (MT) is defined as transfusing three or more units of red blood cells within one hour. A volume of one unit of the red blood cells may be 200 to 240 ml, and upon transfusing one unit of the red blood cells, a hemoglobin level of the patient (10) may increase by approximately 1 g/dl.

The massive blood transfusion prediction unit (140) defines and predicts the necessity for the massive blood transfusion as a variable MT, and may include a deep learning-based prediction model that predicts intraoperative MT within 10 minutes.

The prediction model was implemented by the following method.

Patients targeted for obtaining training data consisted of 18,135 patients who underwent surgery at Seoul National University Hospital (SNUH) and 621 patients who underwent surgery at Boramae Medical Center (BMC) for external validation.

Massive blood transfusion (MT) was defined as administering three or more units of red blood cells within one hour, and cases where information on the timing of transfusion was inaccurate or the amount of transfusion did not meet the definition of massive blood transfusion (MT) were excluded.

In selection and preprocessing of data, preoperative variables such as demographic information of patients, complications, and preoperative test results from electronic health records were collected to develop a preoperative prediction model, and several intraoperative parameters were collected as follows to develop an intraoperative prediction model.

1) features of PPG and derivative variables (VPG, APG), 2) features of intra-arterial blood pressure, 3) NIBP (non-invasively measured blood pressure), and 4) intraoperative hematocrit levels obtained during surgery.

Since the features have been described in detail in the description of the feature extraction unit (130), redundant descriptions will be omitted.

An observation window and a prediction period for said features are set. Intraoperative features extracted from a single 10-minute observation window were used as an input for the prediction model. The observation window was set differently depending on patients who received massive blood transfusion (MT) (experimental group) and patients who did not (control group) (refer to (c) of FIG. 4).

In the experimental group, the intraoperative features were extracted 20 to 10 minutes before the start of massive blood transfusion (MT). Through this method, an important indicator for predicting the necessity for massive blood transfusion (MT) can be captured. In the control group, where massive blood transfusion did not occur during surgery, a randomly selected period throughout the entire surgical duration was extracted as the observation window. This approach is intended to provide a consistent and unbiased sampling method across all non-critical periods. Additionally, to compare optimal prediction times, prediction periods were set differently to 10 minutes, 15 minutes, and 20 minutes before the massive blood transfusion for comparison.

The prediction model is an artificial intelligence prediction model, which calculates the necessity (risk) for massive blood transfusion 10 minutes later during surgery to derive a massive blood transfusion variable, i.e., an MT variable. The prediction model may receive the following data as an input (refer to (d), (e), and (f) of FIG. 4)

Even when only non-invasive monitoring information using 1) features of PPG and 2) intraoperative hematocrit levels obtained during surgery was used as the input, it was possible to predict massive blood transfusion with sufficient accuracy (AUROC=0.96). It was confirmed that sufficiently accurate prediction could be performed compared to a prediction model (AUROC=0.96) that additionally receives features of intra-arterial blood pressure measured through an invasive method (refer to FIG. 6).

The prediction model can calculate the MT risk after 10 minutes. Since such algorithms can learn complex hierarchical and time-series representations, deep learning-based algorithms such as a Recurrent Neural Network (RNN), a Long Short-Term Memory (LSTM), and a Gated Recurrent Unit (GRU) may be used to learn these features.

Meanwhile, as a comparative example, it can be seen that satisfactory results were not obtained (AUROC=0.78) when preoperative variables, such as demographic information, complications, and preoperative laboratory test results, were used as input data. Here, algorithms such as Logistic Regression (LR), Random Forest (RF), XGBoost (XGB), and Light Gradient Boosting Machine (LGBM) were used for the preoperative prediction model (refer to FIG. 6).

The notification unit (150) may notify the medical staff (20) of the necessity for massive blood transfusion directly or through other monitoring equipment (30) when the necessity for massive blood transfusion within a specific time is predicted by the massive blood transfusion prediction unit (140). For example, a warning display regarding the necessity for massive blood transfusion may be displayed on a display device, or a warning sound or an announcement message regarding the necessity for massive blood transfusion may be output through a speaker. Accordingly, it is possible to make the medical staff focusing on surgery immediately aware of the necessity for massive blood transfusion and to allow for appropriate preparation, thereby improving surgical safety.

The other monitoring equipment (30) is a device for monitoring various conditions of the patient, and can detect the necessity for massive blood transfusion in real time by using information received through the notification unit (150) along with various vital signs of the patient.

FIG. 2 is a flowchart for explaining a method for training a massive blood transfusion prediction system through intraoperative blood monitoring according to an embodiment of the present invention.

Referring to FIG. 2, the method for training the massive blood transfusion prediction system includes a data collection step (S100), a data processing step (S200), a model training step (S300), and a validation step (S400).

In the data collection step (S100), data of patients during surgery are collected to obtain training data for the system for predicting massive blood transfusion.

In selecting the patient data, preoperative variables such as demographic information, complications, and preoperative test results from electronic health records are collected to develop a preoperative prediction model, and various intraoperative parameters are collected as follows to develop an intraoperative prediction model.

Meanwhile, for model training, features extracted from plethysmography collected at 500 Hz and intraoperative hematocrit were additionally used, and i) an area under arterial waveform (AUAW) for each heartbeat, ii) mean arterial pressure (MBP), iii) systolic blood pressure (SBP), iv) diastolic blood pressure (DBP), and v) heart rate (HR) may be extracted from the measured intra-arterial blood pressure. In addition, hematocrit is measured according to a determination by an anesthesiologist, and non-invasive blood pressure (NIBP) was measured and utilized as a feature or for comparison.

1) Features of PPG and derivative variables (VPG, APG), 2) features of intra-arterial blood pressure, 3) NIBP (non-invasively measured blood pressure), and 4) intraoperative hematocrit levels (hct) obtained during surgery.

Since the features have been described in detail in the description of the feature extraction unit of FIG. 1, redundant descriptions will be omitted.

In the data processing step (S200), the PPG waveform and derivative variables thereof may be pre-processed using a Savitzky-Golay filter before feature extraction. Features are extracted from the pre-processed data, and specifically, the following features may be extracted from each data.

Features extracted from the PPG waveform include i) a systolic peak(S), ii) a start of a systolic wave (O), iii) (⅓)(S+(2*O))*(M), iv) a cycle duration (Tc), and v) an area under the waveform (au_W).

In the VPG waveform, i) a maximum slope during contraction (w) and ii) a minimum slope during contraction (y) may be extracted as the features.

Since all major points of the VPG waveform reflect vascular conditions, features for i) an a-wave (a), 2) a b-wave (b), 3) a c-wave (c), and 4) a d-wave (d) may be extracted from the VPG waveform.

In the model training step (S300), the features extracted in the data processing step (S200) may be used to train the prediction model of the system for predicting massive blood transfusion. By setting a single 10-minute observation window for the features, the intraoperative features extracted from the single 10-minute interval observation window may be used as an input for the prediction model. The observation window may be set differently depending on whether the patient received massive blood transfusion (MT) (experimental group) or did not receive massive blood transfusion (MT) (control group).

In the validation step (S400), prediction performance of the prediction model trained through various methods may be measured and validated for each feature combination by calculating an area under the receiver operating characteristic curve (AUROC).

To design an optimal prediction model, the AUROCs of the preoperative prediction model and the intraoperative prediction model using various features and input variables were compared using the DeLong test. Sensitivity, specificity, positive predictive value (PPV), negative predictive value (NPV), and the F1 score at an optimal threshold that maximizes the sum of sensitivity and specificity (i.e., Youden's J statistic) were used to predict the performance of the proposed method. The optimal threshold for classification was determined using Youden's J statistic within an internal validation set. Subsequently, the performance of the model was evaluated in an external validation set using the defined threshold.

A 95% confidence interval (CI) of the AUROC was calculated using 2,000 bootstrapping resamplings, and such a risk score was defined as a massive blood transfusion index (MTi). All analyses were performed using R (version 4.3.1) and Python (version 3.8.10).

Specifically, the patient data used for the experiment include patients who underwent surgery at Seoul National University Hospital (SNUH) between August 2016 and December 2019. Among them, patients with intra-arterial blood pressure monitoring records (n=24,686) were included (refer to FIG. 8). The following surgical cases were excluded from the analysis: 1) patients under 18 years of age, 2) patients with low weight (less than 30 kg), 3) patients who received intraoperative transfusion but not MT, 4) patients with short surgery time (less than 20 minutes), and 5) patients without PPG or non-invasive blood pressure (NIBP) monitoring information or with poor signal quality. Patients who underwent surgery at SNUH were randomly divided into 80% for model development and 20% for model validation. For external validation, patients with intra-arterial blood pressure monitoring records (n=746) at Boramae Medical Center (BMC) between 2020 and 2021 were identified. The same exclusion criteria as SNUH were applied to BMC.

FIG. 3 is a flowchart for explaining a massive blood transfusion prediction method using a massive blood transfusion prediction system through intraoperative blood monitoring according to an embodiment of the present invention.

Referring to FIG. 3, the massive blood transfusion prediction method using a massive blood transfusion prediction system through intraoperative blood monitoring can predict the necessity for massive blood transfusion within a predetermined time based on real-time intraoperative monitoring data using a pre-trained prediction model, and notify the medical staff.

The method for predicting massive blood transfusion through intraoperative blood monitoring may include a bio-signal measurement step (S500), a preprocessing step (S600), a feature extraction step (S700), a massive blood transfusion prediction step (S800), and a notification step (S900).

In the bio-signal measurement step (S500), a photo-volumetric pulse wave of a patient may be non-invasively measured through skin of the patient using a photoplethysmogram (PPG) measurement sensor.

In the preprocessing step (S600), a photoplethysmogram (PPG) waveform representing a photoplethysmogram over time may be generated from a signal measured through the PPG measurement sensor, and noise removal or smoothing may be performed.

In the feature extraction step (S700), one or more features may be extracted from the preprocessed PPG waveform.

In the massive blood transfusion prediction step (S800), the necessity for transfusion is predicted by generating a massive blood transfusion index that numerically represents the necessity for massive blood transfusion through the pre-trained prediction model from the features.

In the notification step (S900), the necessity for the massive blood transfusion within 10 to 20 minutes is notified through a change in the massive blood transfusion index generated in the massive blood transfusion prediction step.

Since specific data processing in each of the steps is substantially the same as the description for FIG. 1 above, redundant descriptions will be omitted.

FIG. 4 is a diagram for explaining data selection (a), preprocessing, feature extraction (c), input source selection (d), modeling (e), and a prediction result (f) of the massive blood transfusion prediction system according to an embodiment of the present invention. FIG. 5 is a diagram for explaining data and performance used for training the massive blood transfusion prediction system according to an embodiment of the present invention. FIG. 6 is a diagram for explaining performance of a prediction model according to features and combinations thereof that can be used in a massive blood transfusion prediction unit of the system for predicting massive blood transfusion according to an embodiment of the present invention. FIG. 7 is a diagram showing an example of a real-time massive blood transfusion index (MTi) of a patient who received a massive blood transfusion during surgery. FIG. 8 is a diagram showing a configuration of actual patient data used in an experimental example of the present invention. FIG. 9 is a diagram showing predictive AUROC at 10 minutes, 15 minutes, and 20 minutes of a prediction model used in an experimental example of the present invention.

Referring to FIGS. 4 to 9, performances of three models (a preoperative prediction model, an invasive intraoperative prediction model, and a non-invasive intraoperative prediction model) were compared. First, the best-performing preoperative prediction model was Random Forest (RF) having an AUROC (area under the receiver operating characteristic curve) of 0.781 (95% CI, 0.729-0.831). Then, performances of the non-invasive intraoperative prediction model and the invasive intraoperative prediction model were evaluated using deep learning-based algorithms. Among various deep learning-based algorithms, Gated Recurrent Unit (GRU)—which showed an AUROC of 0.787(95% CI, 0.736-0.837) for PPG features, 0.731 (95% CI, 0.671-0.786) for VPG features, 0.721(95% CI, 0.663-0.767) for APG features, and 0.870 (95% CI, 0.829-0.907) for intra-arterial blood pressure features—was the best algorithm for all intraoperative feature combinations. In internal validation, hct (hematocrit) showed the best performance as a single variable with an AUROC of 0.918(95% CI, 0.883-0.949). In the invasive intraoperative prediction model, the best combination consisted of AUAW and hct, and the AUROC was 0.962(95% CI, 0.942-0.979). In the non-invasive intraoperative prediction model, the best combination consisted of Tc and hct, and the AUROC was 0.962(95% CI, 0.948-0.974). The performance of external validation was maintained with an AUROC of 0.922(95% CI, 0.882-0.959). The AUROCs of the preoperative and non-invasive intraoperative prediction models were significantly different (p<0.001). Furthermore, the non-invasive prediction model was not statistically different from the invasive prediction models of previous studies. In addition, whether the best prediction model can predict MT risk not only 10 minutes before but also 15 or 20 minutes before was investigated, and the performance of the non-invasive intraoperative prediction model according to the prediction period was similar in internal and external validation (10 minutes before vs. 15 or 20 minutes before) (refer to FIG. 9). The developed prediction model not only shows robust performance in predicting MT necessity but also can be effectively extended to broader transfusion requirements.

Meanwhile, since the prediction model shows sufficient predictability even with only the cycle duration (Tc) extracted from the PPG waveform, it may be implemented by simply using only Tc as a feature, which is an input value of the prediction model. Here, the cycle duration (Tc) is a time interval between start and end points of an area under waveform (au_W), and can be calculated using a known calculation method for calculating an area under a waveform from a waveform.

In the internal validation set, there was a 17.6-fold difference in the average MTi value between patients who received intraoperative MT and those who did not. Additionally, the difference between the two groups was 8.27 times greater in the external validation dataset. In most of the control group (patients who did not receive intraoperative MT), MTi was less than 0.01, but in most of the experimental group (patients who received intraoperative MT), MTi was >0.01. Referring to FIG. 7 together, FIG. 7 shows an example of MTi of a patient who received MT at 216 minutes after the start of surgery. The MTi increased above the MT threshold from 162 minutes after surgery.

According to embodiments of the present invention, a deep learning-based algorithm (DLA) that predicts intraoperative MT within 10 minutes using intraoperative bio-signal waveforms non-invasively using PPG can be implemented.

In various prediction models according to embodiments of the present invention, the best-performing DLA includes Tc and hct. PPG is a non-invasive optical technology commonly used to measure changes in blood volume in peripheral tissues. By analyzing PPG signals, important changes such as heart rate (HR) and blood pressure (BP) variations can be predicted. HR can be accurately estimated by analyzing PPG waveform characteristics including amplitude, pulse width, and shape. By applying machine learning algorithms to PPG data, a model capable of predicting HR changes in real time can be trained.

Similarly, PPG signals can provide insights into BP fluctuations by examining pulse transit time (PTT) derived from PPG and other factors such as arterial stiffness. PTT is the time required for an arterial pulse wave to propagate between two arterial sites and is inversely proportional to BP. Through continuous monitoring of PPG signals and the application of advanced data analysis techniques such as signal processing and deep learning, PPG-based prediction models can provide valuable information on critical changes such as the necessity for massive blood transfusion. These predictions can help in early detection of abnormalities and provide feedback for health monitoring and management.

In particular, according to embodiments of the present invention, computational efficiency can be increased and a response time of the model can be shortened by using relatively small data through a non-invasive method.

Although the present invention has been described with reference to the above embodiments, it will be understood by those skilled in the art that various modifications and changes can be made to the present invention without departing from the spirit and scope of the present invention as set forth in the following claims.

### DESCRIPTION OF THE SIGN

149100: massive blood transfusion prediction system
110: PPG sensor
120: waveform generation unit
130: feature extraction unit
140: massive blood transfusion prediction unit
150: notification unit

1. A massive blood transfusion prediction system through intraoperative blood monitoring comprising:
a photoplethysmogram (PPG) measurement sensor configured to non-invasively measure a photo-volumetric pulse wave of a patient through skin of the patient;
a waveform generation unit configured to generate a photoplethysmogram (PPG) waveform representing a photoplethysmogram over time from a signal measured by the PPG measurement sensor;
a feature extraction unit configured to extract one or more features from the PPG waveform; and
a massive blood transfusion prediction unit configured to generate a massive blood transfusion index numerically representing a necessity for massive blood transfusion through a pre-trained prediction model from the features,
wherein the massive blood transfusion is defined as transfusing three or more units of red blood cells within one hour.

2. The massive blood transfusion prediction system according to claim 1, further comprising an notification unit configured to notify whether the massive blood transfusion is required within 10 to 20 minutes through a value of the massive blood transfusion index.

3. The massive blood transfusion prediction system according to claim 1, wherein the feature extracted by the feature extraction unit is a cycle duration (Tc) of the PPG waveform.

4. The massive blood transfusion prediction system according to claim 3, wherein the cycle duration is calculated as a time interval between start and end points in a graph for an area under the PPG waveform.

5. The massive blood transfusion prediction system according to claim 1, wherein the waveform generation unit further generates a velocity of photoplethysmogram (VPG) waveform, which is a first derivative of the PPG waveform,
the feature extraction unit further extracts one or more features from the VPG waveform, and
the massive blood transfusion prediction unit uses two or more features among the features extracted from the PPG waveform and the VPG waveform as an input for the prediction model.

6. The massive blood transfusion prediction system according to claim 1, wherein the waveform generation unit further generates an acceleration of photoplethysmogram (APG) waveform, which is a second derivative of the PPG waveform,
the feature extraction unit further extracts one or more features from the APG waveform, and
the massive blood transfusion prediction unit uses two or more features among the features extracted from the PPG waveform and the APG waveform as an input for the prediction model.

7. A method for training a massive blood transfusion prediction system that predicts in advance a necessity for massive blood transfusion during surgery using a prediction model, the method comprising:
a data collection step of collecting training data including whether and when a massive blood transfusion was performed on a patient during surgery and a PPG waveform;
a data processing step of preprocessing the PPG waveform of the data and extracting features;
a model training step of training the prediction model using the extracted features; and
a validation step of validating performance of the prediction model by calculating an area under the receiver operating characteristic curve (AUROC).

8. The method for training a massive blood transfusion prediction system according to claim 7, wherein the features include at least one of: i) a systolic peak(S), ii) a start of a systolic wave (O), iii) (⅓)(S+(2*O))*(M), iv) a cycle duration (Tc), and v) an area under the waveform (au_W) of the preprocessed PPG waveform.

9. The method for training a massive blood transfusion prediction system according to claim 7, wherein, in the data processing step, a velocity of photoplethysmogram (VPG) waveform, which is a first derivative of the preprocessed PPG waveform, is further generated, and one or more features are further extracted from the VPG waveform, and wherein, in the model training step, training is performed by using two or more features among the features extracted from the PPG waveform and the VPG waveform as an input for the prediction model.

10. The method for training a massive blood transfusion prediction system according to claim 7, wherein, in the data processing step, an acceleration of photoplethysmogram (APG) waveform, which is a second derivative of the preprocessed PPG waveform, is further generated, and one or more features are further extracted from the APG waveform,
and wherein, in the model training step, training is performed by using two or more features among the features extracted from the PPG waveform and the APG waveform as an input for the prediction model.

11. A massive blood transfusion prediction method using a massive blood transfusion prediction system through intraoperative blood monitoring, comprising:
a bio-signal measurement step of non-invasively measuring a photo-volumetric pulse wave of a patient through skin of the patient using a photoplethysmogram (PPG) measurement sensor;
a preprocessing step of generating a photoplethysmogram (PPG) waveform representing a photoplethysmogram over time from a signal measured through the PPG measurement sensor and performing noise removal or smoothing;
a feature extraction step of extracting one or more features from the preprocessed PPG waveform; and
a massive blood transfusion prediction step of generating a massive blood transfusion index numerically representing a necessity for massive transfusion through a pre-trained prediction model from the features,
wherein the massive blood transfusion is defined as transfusing three or more units of red blood cells within one hour.

12. The massive blood transfusion prediction method according to claim 11, further comprising a notification step of notifying a necessity for the massive transfusion within 10 to 20 minutes through a change in the massive blood transfusion index generated in the massive blood transfusion prediction step.

13. The massive blood transfusion prediction method according to claim 11, wherein the feature extracted in the feature extraction step is a cycle duration (Tc) of the PPG waveform.

14. The massive blood transfusion prediction method according to claim 13, wherein the cycle duration is calculated as a time interval between start and end points in a graph for an area under the PPG waveform.

15. The massive transfusion prediction method according to claim 11, wherein, in the preprocessing step, a velocity of photoplethysmogram (VPG) waveform, which is a first derivative of the PPG waveform, or an acceleration of photoplethysmogram (APG) waveform, which is a second derivative of the PPG waveform, is further generated,
wherein, in the feature extraction step, one or more features are further extracted from the VPG waveform or the APG waveform, and
wherein, in the massive blood transfusion prediction step, two or more features among features extracted from the PPG waveform and the VPG waveform, or from the APG waveform, are used as an input for the prediction model.
